(19) United States
(12) Patent Application Publication
(10) Pub. No.: US 2007/0060939 A1
(43) Pub. Date: Mar. 15, 2007

(54) Expandable and retractable cannula

(75) Inventors: Mike E. Lancial, St. Louis Park, MN (US); Madhavi N. Deshpande, Golden Valley, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/218,873

(22) Filed: Sep. 2, 2005

(51) Int. Cl. A61M 29/00

(52) U.S. Cl. 606/191

(57) ABSTRACT

An expandable cannula and method for using the expandable cannula to perform minimally invasive, percutaneous surgeries to access the spine or other bone structures, organs, or locations of the body. The expandable cannula includes a tubular body movable between first and second size states and an actuating mechanism on the tubular body for moving the tubular body between the first and second size states.

### TECHNICAL FIELD

The invention relates generally to devices and methods for performing minimally invasive, percutaneous surgeries. More particularly, the invention is an expandable and retractable cannula.

### BACKGROUND

Traditional surgical procedures often require a long incision, extensive muscle stripping, and prolonged retraction of tissues to access the desired surgical site as well as denervation and devascularization of surrounding tissue. This is particularly the case with spinal applications because of the need for access to locations deep within the body. Such surgical procedures can cause significant trauma to intervening tissues and potential damage to good tissue due to the amount and duration of tissue retraction, resulting in increased recovery time, permanent scarring, and pain that can be more severe than the pain that prompted the original surgical procedure. This is further exacerbated by the need to make a large incision so that the surgeon can properly view the areas inside the body that require attention.

Endoscopic, or minimally invasive, surgical techniques allow a surgical procedure to be performed on a patient's body through a smaller incision in the body and with less body tissue disruption. Endoscopic surgery typically utilizes a tubular structure known as a cannula (or portal) that is inserted into an incision in the body. A typical cannula is a fixed diameter tube, which a surgeon uses to hold the incision open and which serves as a conduit extending between the exterior of the body and the local area inside the body where the surgery is to be performed. Thus, cannulae can be used for visualization, instrument passage, and the like.

The typical cannula, however, presents at least two disadvantages. First, insertion of the cannula typically requires an incision the full depth and diameter of the cannula. Although this incision is often relatively smaller than incisions made for surgical procedures performed without a cannula, there is still trauma to healthy tissue. Additionally, endoscopic surgical techniques may be limited by the size of the cannula because some surgical instruments, such as steerable surgical instruments used in posterior discectomies, are sometimes larger than the size of the opening defined by the cannula. Therefore, there is a need for a cannula that can be inserted with minimal incision of tissue yet still provide an entrance opening and conduit sized for sufficient instrument passage and operation.

### SUMMARY

The invention is generally directed to a device and method for performing minimally invasive, percutaneous surgeries to access the spine or other bone structures, organs, or locations of the body. In one embodiment, the invention is an expandable cannula having a tubular body that may be moved between a first, or expanded, size state and a second, or relatively reduced, size state. An actuating mechanism on the tubular body can be actuated to move the tubular body between the first and second size states. In one embodiment, the actuating mechanism is actuated to move the tubular body between the first and second size states, thereby increasing the size at a distal end of the tubular body. In an alternative embodiment, the size at the distal end of the tubular body is decreased by actuating the actuating mechanism to move the tubular body between the first and second size states.

Another embodiment of the invention is a method for using the expandable and retractable cannula to access an internal body part.

### BRIEF DESCRIPTION OF THE DRAWINGS

Other objects and advantages of the invention will become apparent upon reading the following detailed description and upon reference to the drawings in which:

FIG. 1 is an illustration of an expandable cannula in accordance with one embodiment of the invention showing the device in an expanded diameter state.

FIG. 2A is an illustration of the expandable cannula shown in FIG. 1 from the top.

FIG. 2B is an illustration of the expandable cannula shown in FIG. 1 from the top according to another embodiment of the invention.

FIG. 3 is an illustration of the expandable cannula shown in FIG. 1 from the bottom.

FIG. 4 is an illustration of an expandable cannula in accordance with one embodiment of the invention showing the device in a reduced size state without a skirt.

FIG. 5 is an illustration of an elongated curvilinear rod-shaped rib for the frame in accordance with one embodiment of the invention.

FIG. 6 is an illustration of an elongated curvilinear paddle-shaped rib for the frame in accordance with an alternative embodiment of the invention.

FIG. 7 is a cross-sectional view of the device shown in FIG. 1 without a skirt showing a plurality of ribs coupled to a rotating member.

FIG. 8 is a detailed view of a gear ring, a gear, and a rib of the device of FIG. 1 in which the rotating member 7 is removed for clarity.

FIG. 9 is a detailed view of a portion of an actuating mechanism for an expandable cannula having individually actuatable ribs in accordance with another embodiment of the invention.

FIG. 10 is an exploded view of the device of FIG. 1.

While the invention is susceptible to various modifications and alternative forms, specific embodiments have been shown by way of example in the drawings and are described herein. It should be understood, however, that the description herein of specific embodiments is not intended to limit the invention to the particular forms disclosed. On the contrary, the intention is to cover all modifications, equivalents, and alternatives falling within the spirit and scope of the invention as defined by the appended claims.

### DETAILED DESCRIPTION

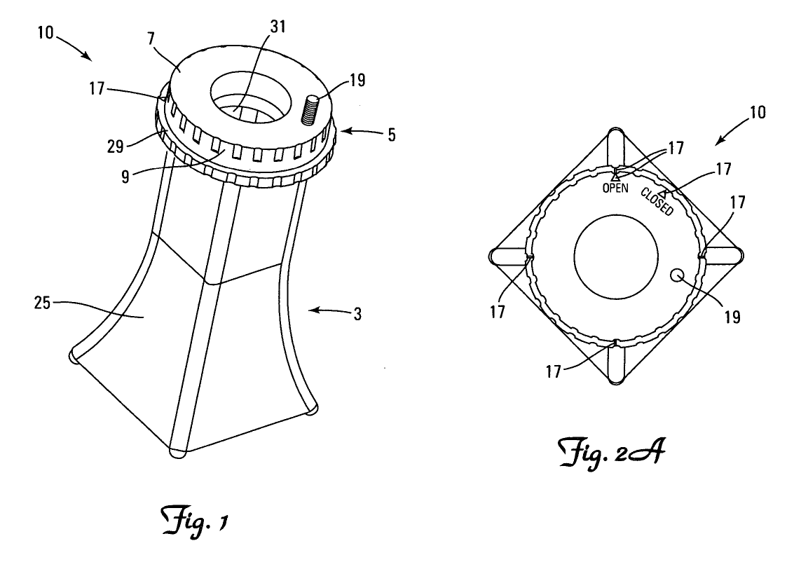

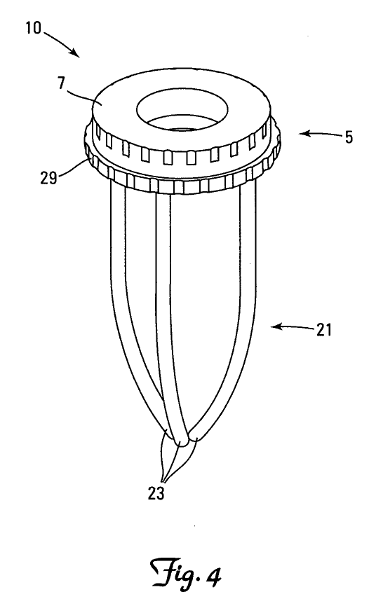

An expandable cannula 10 in accordance with one embodiment of the invention is shown in FIGS. 1 and 4. As shown, the expandable cannula 10 includes an actuating mechanism 5 on a tubular body 3. The actuating mechanism 5 can be actuated to move the tubular body 3 between a first, or expanded, size state and a second, or relatively reduced, size state. FIG. 1 illustrates the expandable cannula 10 in a first, or expanded, size state while FIG. 4 illustrates the expandable cannula 10 in a second, or relatively reduced, size state. As is shown in FIGS. 1 and 4, in the first, or expanded, size state, the tubular body 3 has a greater cross-sectional area than in the second, or reduced, size state.

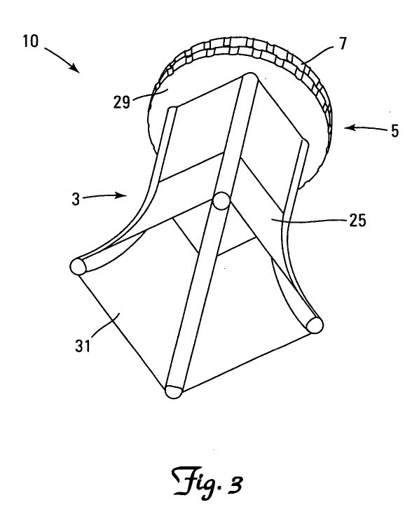

The illustrated embodiment of the tubular body 3 is connected at a proximal end to the actuating mechanism 5 and includes a frame 21 formed by ribs 23 and a skirt 25 supported by the ribs 23. Together with the frame 21 and the skirt 25, the tubular body 3 defines a conduit 31 as shown in FIGS. 1 and 3 through which the surgeon may view the area of interest or insert surgical instruments by providing a barrier against surrounding tissue, organs, bodily fluids, and the like.

The illustrated embodiment of the actuating mechanism 5 includes a rotating member 7 having a textured or roughened perimeter surface 9 to better grip the rotating member 7. The illustrated embodiment of the actuating mechanism 5 also includes a stationary member 29 connected to the rotating member 7.

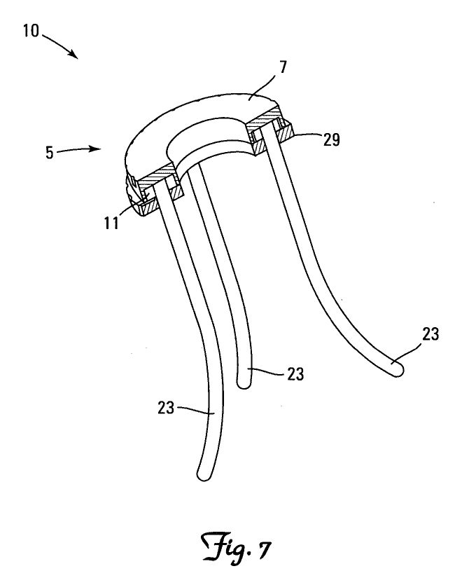

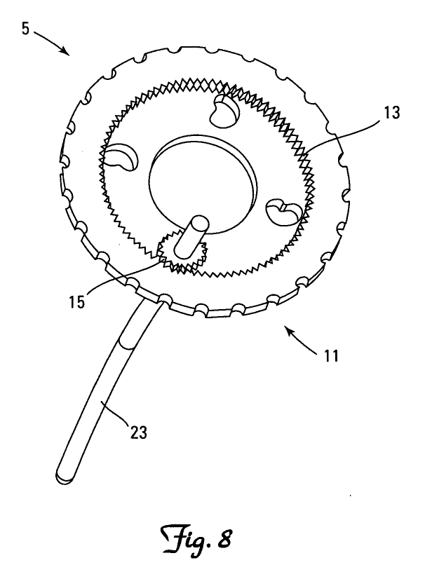

As shown in FIGS. 7 and 8, the actuating mechanism 5 further includes a mechanical linkage 11 for coupling the rotating member 7 to the tubular body 3 and for moving the tubular body 3 between the first and second size states in response to rotation of the rotating member 7.

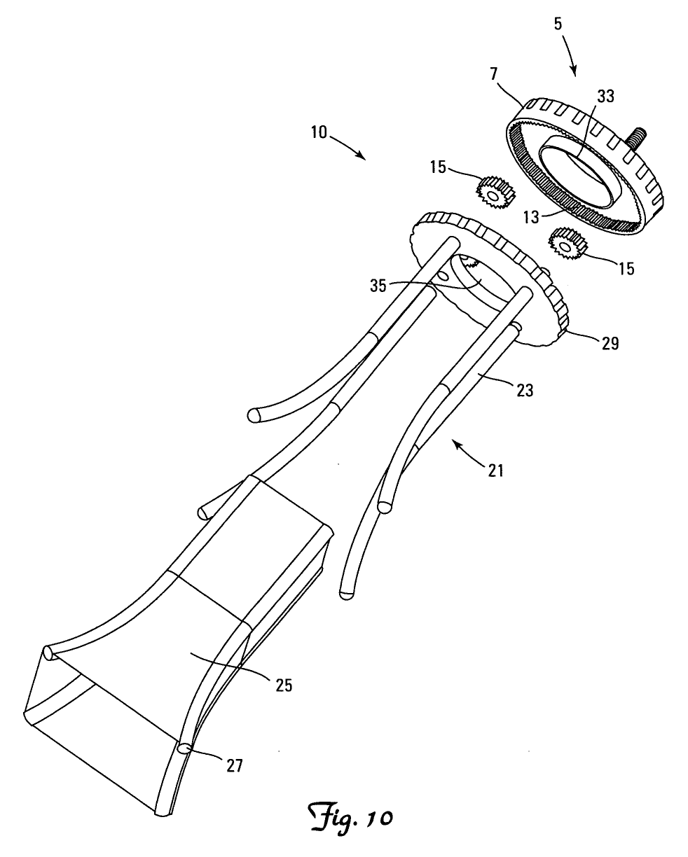

The ribs 23 of the frame 21 are elongated curvilinear members positioned about a circumference of the tubular body 3 in the embodiment shown in FIG. 10. Generally, the circumference of the tubular body is circular but other, non-circular configurations are also contemplated by the invention. These non-circular configurations may include shapes such as an oval, triangle, rectangle, as well as other, less structured, shapes desired to provide access to the surgical site.

The quantity of ribs 23 used to form the frame 21 can be varied to best suit the intended application of the expandable cannula 10. For example, in the illustrated embodiment, the frame 21 is formed by four ribs 23 (the fourth rib 23 is hidden by the skirt 25). In other embodiments (not shown), the frame 21 is formed by three ribs 23, 5 ribs 23 or a greater number of ribs 23 as desired.

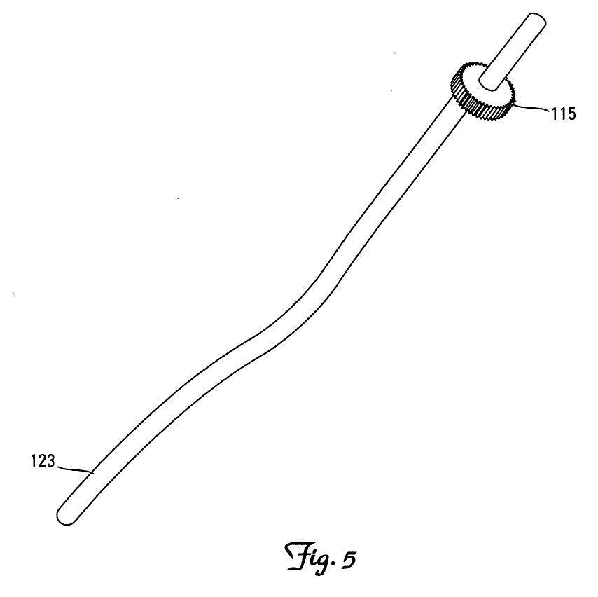

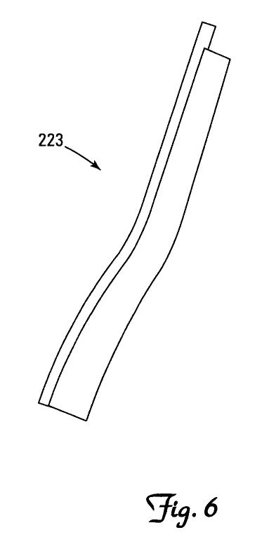

The shape of the ribs 23 used to form the frame 21 can also be varied to best suit the intended application of the expandable cannula 10. For instance, FIG. 5 shows a rib 23 assuming the shape of an elongated curvilinear rod. Alternatively, FIG. 6 shows a rib 23 assuming the shape of an elongated curvilinear paddle. Other shapes, such as a bell shape, that would be suitable to optimize the particular application of the expandable cannula 10 are also contemplated by the invention.

The ribs 23 are generally bowed as shown in FIGS. 5 and 6. By actuating the actuating mechanism 5, these ribs 23 are rotated such that the distal ends of the ribs 23 extend outwardly to increase the size or the cross-sectional area of the tubular body 3 at a distal end of the expandable cannula 10 while maintaining a constant size at a proximal end of the expandable cannula 10. These ribs 23 can also be rotated such that the distal ends of the ribs 3 extend inwardly to decrease the size of the tubular body 3 at the distal end of the expandable cannula 10 while maintaining a constant size at a proximal end of the expandable cannula 10. It is also contemplated that actuation of the actuating mechanism 5 can cause the ribs 23 to rotate such that at least one of the distal ends of the ribs 23 extends inwardly and at least one of the distal ends of the ribs 23 extends outwardly, thereby allowing for localized expansion from one side of the expandable cannula 10 and not the other, for example. It is further contemplated that actuation of the actuating mechanism 5 can cause the ribs 23 to rotate such that tubular body 3 has varying sizes or cross-sectional areas along the length of an intermediate region the tubular body 3 between the proximal and distal ends.

The overall length of the ribs 23 can be set to suit the particular application and the anatomy of the surgical site. Accordingly, various lengths are contemplated by the invention. In one embodiment, the overall length of the ribs 23 is about 5 inches. In another embodiment, the overall length of the ribs 23 is about 7 inches. In yet another embodiment, the overall length of the ribs 23 is about 9 inches.

The ribs 23 can generally be constructed of biocompatible materials that are sufficiently strong and resilient to withstand pressure exerted by the surrounding tissue and body parts. Examples of suitable materials include metals, such as a surgical stainless steel, and shape memory alloys, such as nitinol, as well as plastics, such as polycarbonate and Delrin®, or other sufficiently strong polymers. In some applications, the ribs 23 are constructed of reusable, durable, sterilizable materials. Alternatively, the ribs 23 are constructed of disposable materials or more lightweight materials.

In one embodiment, the ribs 23 are detachable from the actuating mechanism 5. Thus, the ribs 23 may be more easily sanitized for later use or replaced with new ribs 23 so that the expandable cannula 10 can be used with a subsequent patient or repaired in the event damage occurs to one of the ribs 23.

In the illustrated embodiment, the skirt 25 is supported by the frame 21 such that actuation of the actuating mechanism 5 causes the skirt 25 to move with the frame 21 between the first and second size states. The skirt 25 is generally wrapped around the perimeter of the frame 21 formed by the ribs 23. In the illustrated embodiment, the skirt 25 has compartments 27, or pockets, for engaging the ribs 23 as shown in FIG. 10. In this embodiment, the ribs 23 are inserted into the compartments 27 along their full length.

The skirt 25 can be secured to the frame 21 at the proximal end of the frame 21 to prevent the skirt 25 from sliding off of the frame 21 during insertion and removal of the expandable cannula 10. For example, the proximal end of the skirt 25 may be adhesively secured to the proximal end of the frame 21. In one embodiment (not shown), the pockets 27 are closed and/or reinforced at a distal end so that the skirt 25 resists being pushed along the ribs 23 toward the proximal end of the frame 21 during insertion of the expandable cannula 10, which would expose portions of the ribs 23 and potentially compromise the conduit 31. Although it is intended that the skirt 25 remain in place during insertion and removal of the expandable cannula 10, the skirt 25 should also be detachable such the skirt 25 is disposable. Alternately, the skirt 25 may be formed of a material that allows it to be sterilized for later, repeated use.

The skirt 25 is preferably constructed of a biocompatible, elastic material that retains a substantially smooth surface when the skirt 25 moves with the frame 21 between the first and second size states. In one embodiment, the skirt 25 is constructed of a material that returns to its original, unstretched form in the absence of mechanical force without wrinkling. In another embodiment, the skirt 25 is provided with pre-stressed or fold lines, for example, pleats, along which the skirt 25 tends to bend when moving between size states. The skirt 25 preferably also resists stretching against forces typically exerted by retracted tissues, body parts, and bodily fluids yet stretches enough against the mechanical force of the frame 21 as it moves between the first and second size states. The skirt 25 can also be constructed of a material that has minimal tendency to grip the surrounding tissue or body parts, which may cause damage to the tissue or body parts. Suitable materials of construction for the skirt 25 include materials such as silicone, latex or of C-Flex®, a general purpose, thermoplastic elastomer sold by Linvatec Corporation, Clearwater, Fla. Other suitable materials include shape memory materials or nitinol. The skirt 25 can also be constructed of an elastic, biodegradable material that may be left in the body to be reabsorbed by the body without damaging tissues in the body. In one embodiment, the biodegradable skirt 25 can further include a coating that has therapeutic benefits, promotes tissue growth, prevents infection, etc. It is also contemplated that the skirt 25 can be constructed of a transparent material for increased visibility.

The actuating mechanism 5 can be actuated by rotating the rotating member 7 in either a clockwise or counterclockwise direction, resulting in movement of the tubular body 3 between the first and second size states. In one embodiment, the rotating member 7 is operated manually but it is also contemplated that other tools and methods can be employed to rotate the rotating member, such as in situations where access to the rotating member is limited or additional leverage is required. The stationary member 29 can also be used for leverage or as a counter force when rotating the rotating member 7 so that actuation of the actuating mechanism 5 does not simply cause the expandable cannula 10 to be shifted from its position within the body. In one embodiment as illustrated by FIG. 10, the rotating member 7 is secured to the stationary member 29 for rotation by a cylindrical lip 33 on the rotating member 7 that extends into a hole 35 in the stationary member 29. A snap-fit or other mechanism can be used to engage the lip 33 with the stationary member 29.

In the illustrated embodiment, the mechanical linkage 11 is a gear system having a plurality of gears 15, each mounted to one of the ribs 23, and a gear ring 13 on the rotating member 7. FIG. 8 is a detailed illustration of a gear ring 13, a gear 15, and a rib 23 of the illustrated embodiment. As shown in FIG. 8, the gear ring 13 is located around an inner perimeter of the rotating member 7. Consistent with the general operation of a gear system, movement of the rotating member 7 in either a clockwise or counterclockwise direction will result in rotation of the gear ring 13 in the same direction, causing the gears 15 to move in the opposite rotational direction. Because the rib 23 is connected to the gear 15, it will move in the same rotational direction as the gear 15, ultimately causing the rib 23 to rotate and the distal end of the rib 23 to move between the first and second size states. In one embodiment, the rib 23 is connected to the center of the gear 15. Alternatively, the rib 23 can be connected to the gear 15 such that the axis of the rib 23 is offset compared to the axis of the gear 15 to facilitate operation of the actuating mechanism 5 as a cam system and generally increase the amount of overall expansion and contraction of the expandable cannula 10. In addition, the opening through which the rib 23 extends may be kidney-shaped such that the rib 23 travels along the opening when operating as a cam system.

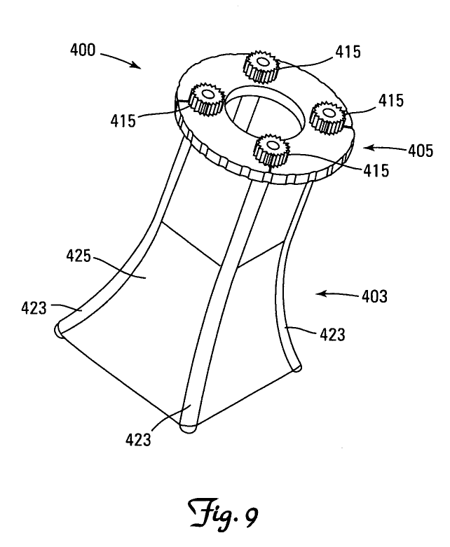

FIG. 9 shows an expandable cannula 400 according to another embodiment of the invention. Cannula 400 involves an actuating mechanism 405 on a tubular body 403. In the present embodiment, the tubular body 403 includes a frame 421 formed by ribs 423 and a skirt 425. In the present embodiment, the ribs 423 are each individually actuatable via a grip 415. In this manner, individual ribs 423 may be rotated to change the size of the tubular body 403 asymmetrically or to move the tubular body 403 between size states one rib 423 at a time.

Although spur gears are illustrated, other gear systems such as bevel gears, gear and belt systems, and cam systems are contemplated by the invention. It is further contemplated that the gears 15 can be of varying diameters to allow for localized expansion from one side of the expandable cannula 10 and not the other, for example. Additionally, the gear ring 13 can be located around an exterior perimeter such that the gears 15 rotate around the outside of the gear ring 13 instead of the inside of the gear ring 13 as shown in FIG. 8 for increased visibility and access to the desired tissue site through the conduit 31.

Suitable materials of construction for the actuating mechanism 5, including the rotating member 7, mechanical linkage 11, gear ring 13, and gears 15, include materials of sufficient strength to be able to withstand the pressure of the tissues being retracted by the expandable cannula 10, such as stainless steel. The actuating mechanism 5 is preferably constructed of materials that can withstand autoclave temperatures, such that they can be sterilized for subsequent use. The actuating mechanism 5 can also be constructed of biocompatible materials such as surgical stainless steel. Other suitable materials of construction can be used in this invention and one of skill in the art could readily select the appropriate materials based upon the intended application. For example, other materials can be used if the actuating mechanism 5 or other parts of the cannula 10 are integrated for single use applications.

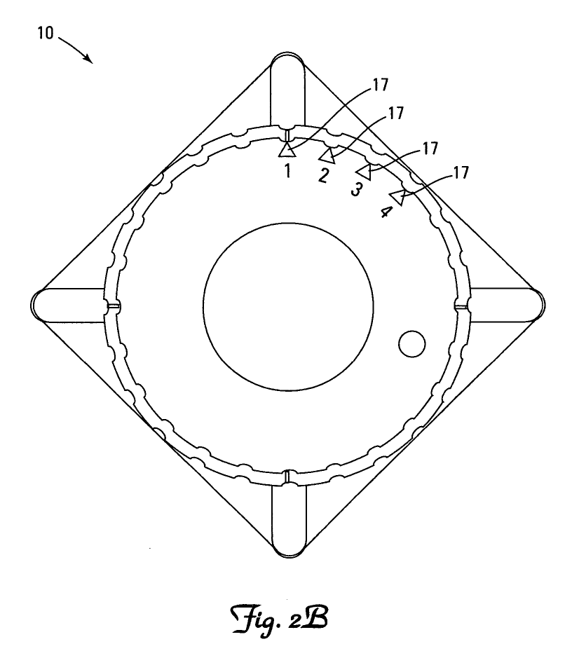

As illustrated in FIGS. 2A and 2B, the actuating mechanism 5 can include indices 17 for monitoring the expansion or contraction of the tubular body 3 as it moves between the first and second size states in response to actuation of the actuating mechanism 5. As seen in FIG. 2A, the indices 17 can be visual markings such as an indexing mark on the stationary member 29 combined with marks on the rotating member 7 that correspond to the first and second size states (i.e., “open” and “close”). Alternately, as shown in FIG. 2B, the indices 17 can correspond to multiple intermediate size states (i.e., 1-4), in which “1” represents the smallest size state and “4” represents the largest size state. Alternatively, the indices 17 can be an audible tone such as a clicking sound that can be heard when the actuating mechanism 5 is actuated. It is contemplated that the audible tone can correspond to whether the tubular body 3 is moving between the first and second size states or has reached the first or second size state. The indices 17 can also be used to monitor the extent of the expansion of the expandable cannula 10 to better prevent tissue damage by over expansion. The indices 17, therefore, can provide particular advantage to the surgeon during the process of “dialing in” to the appropriate size state, making it easier for the surgeon to adapt the expandable cannula 10 to the particular application.

As is shown in FIGS. 1 and 2, the actuating mechanism 5 can further include a locking device 19. In one embodiment, the locking device 19 is connected to the proximal end of the tubular body 3 to restrict the tubular body 3 from moving between the first and second size states. Optionally, the locking device 19 is operable to restrict or to lock in position individual ribs 23 while permitting actuation of other ribs 23 (such as for use with the embodiment of the invention shown generally in FIG. 9). A suitable locking device 19 is a pin that obstructs movement actuating mechanism 5 as well as the tubular body 3. Suitable materials of construction for the locking device 19 include materials of sufficient strength to be able to withstand the pressure of the tissues being retracted by the expandable cannula 10. The materials can also include biocompatible and/or sterilizable materials such as stainless steel.

The invention is not limited to particular sizes for the conduit 31, or the first and second size states, because the actual dimensions of the expandable cannula 10 will depend upon the anatomy of the surgical site and the type of surgery being performed. Accordingly, various sizes are provided by the invention. In one embodiment, the size of the first and second size states can vary between about 19 mm to 25 mm. In another embodiment, the size of the first and second size states can vary between about 19 mm to 40 mm. In the illustrated embodiment, the proximal end of the expandable cannula 10 will retain a constant size. In a particular embodiment, this size is about 19 mm. In other embodiments, the proximal end of the expandable cannula 10 has varying size states. It is further contemplated that the expandable cannula 10 can be continuously adjusted to allow the surgeon to “dial in” to the appropriate first or second size state. Thus, the surgeon may incrementally increase the size or cross-sectional area at the distal end of the expandable cannula 10, permitting the surrounding body tissues to slowly stretch, adapt to the new position, and relax.

The invention has many uses in the surgical field including the spinal surgical field, specifically percutaneous surgical procedures such as laminotomies, laminectomies, foramenotomies, facetectomies, or discectomies. It is also contemplated that the invention may be used for other surgical applications, particularly where minimally invasive surgical fields and still other applications are desired.

The invention additionally provides a method of accessing an internal body part by using the expandable cannula 10 previously described. The method includes the step of forming an opening in the body, such as by an incision in the epidermis. The expandable cannula 10 is then inserted into the body through the opening in a contracted state. In some embodiments, the opening is formed through blunt dissection and the cannula 10 is guided into the opening with the surgeon's fingers. In this embodiment, the cannula 10 is its own dilator. In other embodiments, the opening is formed at more shallow angles and the cannula 10 is guided into the opening with a separate dilator, and possibly a guide wire. With the expandable cannula 10 in position, the conduit 31 forms a working channel. In some instances, it may also be desirable to hold the expandable cannula 10 in place by use of a fixation device such as a mounting bracket attached to a flexible support arm (not shown). This fixation device can be readily adjusted into a fixed position to support for the expandable cannula 10 and provide the surgeon with increased accessibility to the conduit 31. The selection of the appropriate fixation device can be readily accomplished by one of skill in the arm.

Following insertion of the contracted expandable cannula 10 into the body, the surgeon can further expose an internal body part by actuating the actuating mechanism 5 of the expandable cannula 10 to cause the tubular body 3 to move between first and second size states. The surgeon can gradually and incrementally actuate the actuating mechanism 5 until a desired size state is obtained. The surgeon can then lock the expandable cannula 10 by activating the locking device 19.

Once the desired size state has been reached, the surgeon can conduct the surgical procedure through the conduit 31 as necessary, including inserting necessary tools and instruments such as standard surgical implements and visualization scopes. Upon completion of the surgical procedure and removal of the tools and instruments, the surgeon can deactivate the locking device 19 and then gradually decrease the size or cross-sectional area at the distal end of the cannula until a desired size state is obtained by actuating the actuating mechanism 5 to cause the tubular body 3 to move between the first and second size states. If the expandable cannula 10 has been attached to a fixation device, the fixation device is removed, allowing the expandable cannula 10 to be removed from the body. The incision may then be closed using standard surgical procedures.

Following use, the expandable cannula 10 can be sanitized for future use, such as by use of an autoclave or other chemical sanitation processes. In one embodiment, the skirt 25 and/or ribs 23 are also detached from the actuating mechanism 5 and either sanitized for future use or replaced with a new skirt 25 and/or ribs 23.

The particular embodiments disclosed above are intended to be illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above can be altered or modified and all such variations are considered within the scope and spirit of the invention. Accordingly, the protection sought herein is as set forth in the claims below.

What is claimed is:

1. An expandable cannula comprising:
a tubular body including a frame formed by a plurality of ribs, each rib having at least a first curved portion, wherein the tubular body is movable between first and second size states; and
an actuating mechanism for moving the tubular body between the first and second size states, wherein the actuating mechanism includes a rotating member and a mechanical linkage connected to the tubular body at a proximal end of the ribs for moving the tubular body between the first and second size states in response to rotation of the rotating member.

2. The expandable cannula of claim 1, wherein the actuating mechanism comprises a stationary member connected to the rotating member for providing a counter force when the rotating member is rotated.

3. The expandable cannula of claim 1, wherein the rotating member comprises a surface for gripping the rotating member.

4. The expandable cannula of claim 1, wherein the mechanical linkage comprises a gear system.

5. The expandable cannula of claim 4, wherein the gear system comprises a plurality of gears connected with a gear ring such that rotation of the gear ring rotates the plurality of gears.

6. The expandable cannula of claim 1, wherein the actuating mechanism comprises indices for monitoring the size state of the tubular body as it is moved between the first and second size states in response to actuation of the actuating mechanism.

7. The expandable cannula of claim 6, wherein the indices are a visual marking or an audible sound.

8. The expandable cannula of claim 1, wherein the actuating mechanism comprises a locking device connected to the proximal end of the tubular body to restrict the tubular body from moving between the first and second size states.

9. The expandable cannula of claim 1, wherein the ribs are elongated curvilinear rods.

10. The expandable cannula of claim 1, wherein the ribs are elongated curvilinear paddles.

11. The expandable cannula of claim 1, wherein the ribs are individually actuatable.

12. The expandable cannula of claim 1, wherein the frame is detachable from the actuating mechanism.

13. The expandable cannula of claim 1 wherein the tubular body further comprises a skirt supported by the frame wherein actuation of the actuating mechanism causes the skirt to move with the frame between the first and second size states.

14. The expandable cannula of claim 13, wherein the skirt is constructed of an elastic material that retains a substantially smooth surface when the skirt moves with the frame between the first and second size states.

15. The expandable cannula of claim 13, wherein the skirt is detachable from the frame.

16. An expandable cannula comprising:
a tubular body having a proximal end and a distal end, wherein the proximal end has a first size state and the distal end is movable between a first size state and a second size state; and
a rotatable actuating mechanism on the tubular body for moving the distal end of the tubular body between the first and second size states while the proximal end remains in the first size state.

17. A method of accessing an internal body part comprising:
forming an opening in the body;
inserting an expandable cannula having a first size state into the body through the opening; and
rotating an actuating mechanism at a proximal end of the expandable cannula to move a distal end of the expandable cannula between the first size state and a second size state while the proximal end of the expandable cannula remains in the first size state.

18. The method of claim 17 wherein moving the distal end of the expandable cannula further comprises selectively moving portions of the distal end of the expandable cannula.

19. The method of claim 17 wherein rotating the actuating mechanism further comprises indicating the movement of the tubular body from the first size state to the second size state in response to rotation of the actuating mechanism.

20. The method of claim 19 wherein indicating the movement of the tubular body includes providing one of visual or audible indices.
